(19) United States
(12) Patent Application Publication
(10) Pub. No.: US 2023/0070634 A1
(43) Pub. Date: Mar. 9, 2023

(54) HANDS-FREE FOOT CARE AND CALLOUS REMOVAL DEVICE

(71) Applicant: Mark J. Henson, Griffin, GA (US)

(72) Inventor: Mark J. Henson, Griffin, GA (US)

(21) Appl. No.: 17/466,221

(22) Filed: Sep. 3, 2021

(51) Int. Cl. A61B 17/54

(52) U.S. Cl. CPC A61B 17/54; A61B 2017/00761

(57) ABSTRACT

A hands-free foot care and callous removal device is provided, configured to enable insertion of a user's foot into the device to contact brush plates for hands-free cleaning and/or massaging of the foot and/or to enable the user to contact abrasive surfaces with the foot's top, sides, bottom, and/or heel for hands-free abrading of dead skin and/or callouses on the foot. A method of using the device for hands-free cleaning, massaging, or abrading the foot is also provided, particularly wherein the user has difficulty accessing surfaces of their feet due to a chronic condition such as arthritis, obesity, chronic back pain, or injury.

### FIELD OF THE INVENTION

The present invention relates to a personal hygiene device, and more particularly to a hands-free foot care and callous removal device.

### BACKGROUND

Many individuals have difficulty accessing surfaces of their feet for cleaning, massaging, or abrading callouses in order to maintain healthy feet. Dry skin and callouses on feet can become cracked and painful. If the cracking is deep enough to start to bleed, it can lead to infection, which is an especially serious problem for anyone with a chronic disease, such as diabetes or a lowered immune system due to age or illness. In such individuals, infections can lead to ulcerations and, if not treated, to amputation.

Individuals with difficulty accessing surfaces of their feet must therefore either obtain help from others to wash and maintain their feet or must use devices that facilitate access to their feet. Some prior art devices designed for this purpose require a user to reach their feet with their hands, which is impossible for many who suffer from conditions such as arthritis, obesity, chronic back pain, or injury. Other prior art foot care devices do not facilitate access to all of the main surfaces of the foot, namely the top, sides, bottom, and heel.

Accordingly, there is a need for improved means for foot care and callous removal that can be used by a person of limited agility to massage or clean their feet or to treat calluses on the main surfaces of their feet.

### SUMMARY

To address the foregoing problems, in whole or in part, and/or other problems that may have been observed by persons skilled in the art, the present disclosure provides compositions and methods as described by way of example as set forth below.

In one embodiment, a hands-free foot care and callous removal device is provided, comprising:
a) a base comprising a top surface, a bottom surface, a substantially horseshoe-shaped portion comprising two opposing side walls 116a and 116b, wherein the substantially horseshoe shaped portion 118 and opposing side walls 116a and 116b comprise abrasive surfaces;
b) a bridge comprising a top surface, a bottom surface, and two ends, wherein each end is attached to the base, and wherein the bottom surface of the bridge and the top surface of the base are separated by a gap;
c) a bottom brush plate; and
d) a top brush plate;
   wherein the gap, the bottom brush plate, and the top brush plate are configured to enable insertion of a user's foot into the gap to contact the bottom brush plate and/or the top brush plate for hands-free cleaning and/or massaging of the foot; and wherein the substantially horseshoe shaped portion and opposing side walls comprising abrasive surfaces are configured to enable the user to contact the abrasive surfaces with the foot's top, sides, bottom, and/or heel for hands-free abrading of dead skin and/or callouses on the foot.

In some embodiments, the top brush plate and the bottom brush plate comprise bristles that are either non-movable (static, rigid, etc.) or movable (flexible, deflectable, bendable, etc.) relative to their points of attachment to the bottom surface of the bridge or the top surface of the base.

In another embodiment, a method is provided for the hands-free use of the foot care and callous removal device described above to treat a user's foot, comprising: i) a user inserting their foot into the gap to contact the bottom brush plate for hands-free cleaning and/or massaging of the foot; and/or ii) a user contacting the abrasive surfaces of the substantially horseshoe shaped portion and opposing side walls with the foot's top, sides, bottom, and/or heel for hands-free abrading of dead skin and/or callouses on the foot. In some aspects, the user has difficulty accessing surfaces of their feet for cleaning, massaging, or abrading callouses due to a chronic condition comprising arthritis, obesity, chronic back pain, or injury.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 is a perspective view of a hands-free foot care and callous removal device in accordance with an embodiment of the invention;

FIG. 2 is a front view of the device of FIG. 1;

FIG. 3 is a back view of the device of FIG. 1;

FIG. 4 is a side view of the device of FIG. 1;

FIG. 5 is a top view of the device of FIG. 1; and

FIG. 6 is a bottom view of the device of FIG. 1.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

### Hands-Free Foot Care and Callous Removal Device

The present invention is directed to a hands-free foot care and callous removal device. The present invention allows a user who has difficulty reaching their feet with their hands to clean and/or massage their feet and/or treat callouses on the main surfaces of their feet, particularly the tops, sides, bottoms, and heels of their feet.

In one embodiment, a hands-free foot care and callous removal device is provided, comprising:
a) a base comprising a top surface, a bottom surface, a substantially horseshoe-shaped portion comprising two opposing side walls, wherein the substantially horseshoe shaped portion and opposing side walls comprise abrasive surfaces;
b) a bridge comprising a top surface, a bottom surface, and two ends, wherein each end is attached to the base, and wherein the bottom surface of the bridge and the top surface of the base are separated by a gap;
c) a bottom brush plate; and
d) a top brush plate;
   wherein the gap, the bottom brush plate, and the top brush plate are configured to enable insertion of a user's foot into the gap to contact the bottom brush plate and/or the top brush plate for hands-free cleaning and/or massaging of the foot; and wherein the substantially horseshoe shaped portion and opposing side walls comprising abrasive surfaces are configured to enable the user to contact the abrasive surfaces with the foot's top, sides, bottom, and/or heel for hands-free abrading of dead skin and/or callouses on the foot.

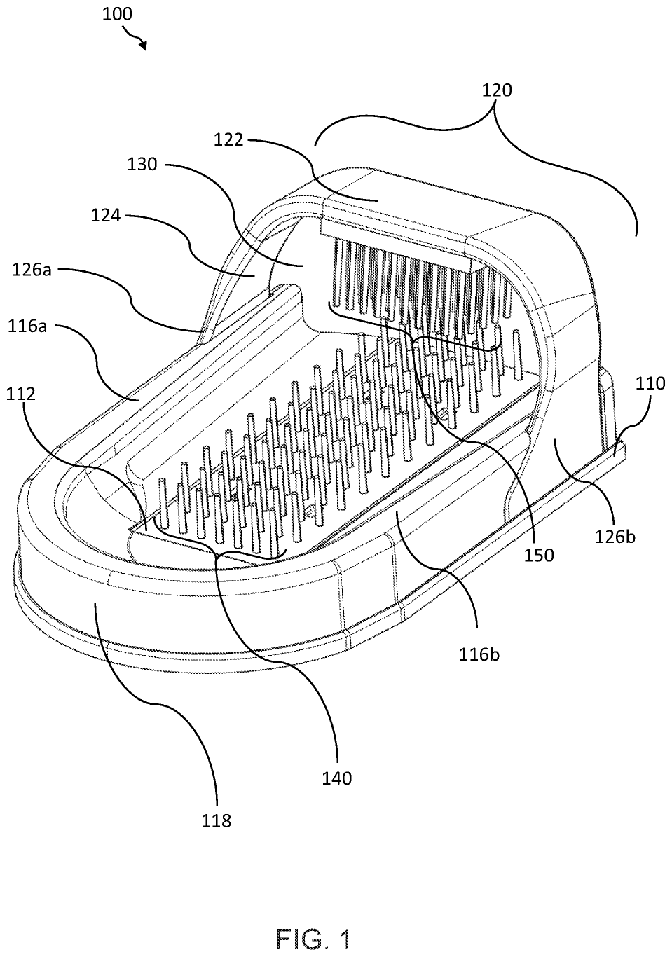

Referring now to FIG. 1, a perspective view of hands-free foot care and callous removal device 100 is provided in accordance with an embodiment of the invention. Foot care and callous removal device 100 comprises:
a) a base 110 comprising a top surface 112, a bottom surface (not shown), and a substantially horseshoe shaped portion 118 comprising two opposing side walls 116a and 116b, wherein the substantially horseshoe shaped portion 118 and opposing side walls 116a and 116b comprise abrasive surfaces;
b) a bridge 120 comprising a top surface 122, a bottom surface 124, and two ends 126a and 126b, wherein end 126a and end 126b are each attached to the base 110, and the bottom surface of the bridge 124 and the top surface of the base 112 are separated by a gap 130;
c) a bottom brush plate 140; and
d) a top brush plate 150;
   wherein the gap 130, the bottom brush plate 140, and the top brush plate 150 are configured to enable insertion of a user's foot into the gap 130 to contact the bottom brush plate 140 and/or the top brush plate 150 for hands-free cleaning and/or massaging of the foot; and wherein the substantially horseshoe shaped portion 118 and opposing side walls 116a and 116b comprising abrasive surfaces are configured to enable the user to contact the abrasive surfaces with the foot's top, sides, bottom, and/or heel for hands-free abrading of dead skin and/or callouses on the foot.

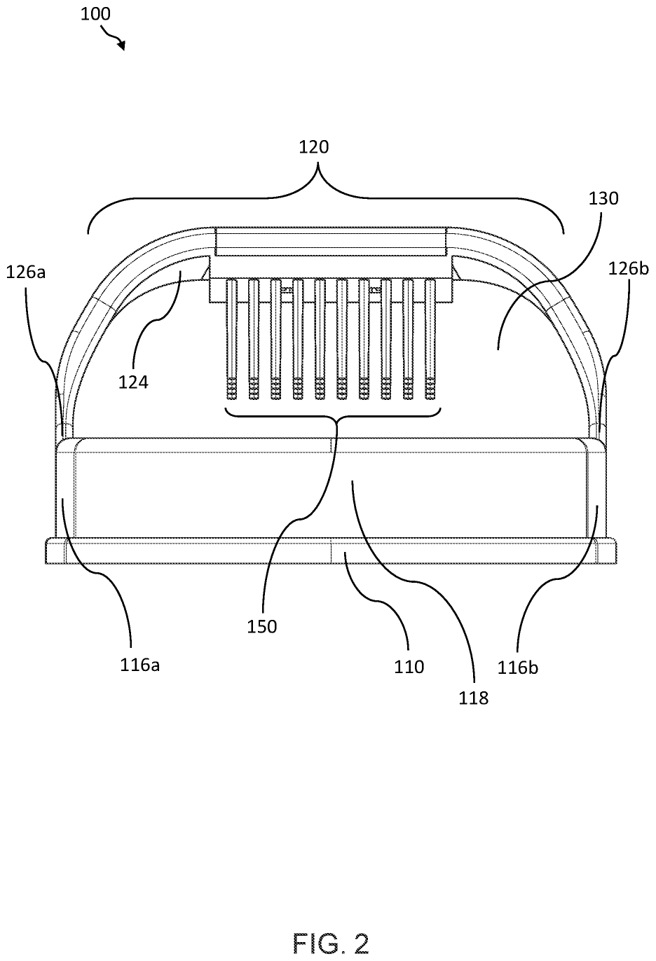

Referring now to FIG. 2, a front view is provided of the device of FIG. 1 showing hands-free foot care and callous removal device 100 comprising base 110; bridge 120 comprising bottom surface 124 and two ends 126a and 126b; the bottom surface of the bridge 124 and the top surface of the base (not shown) separated by gap 130; a top brush plate 150; and a substantially horseshoe shaped portion 118 comprising two opposing side walls 116a and 116b.

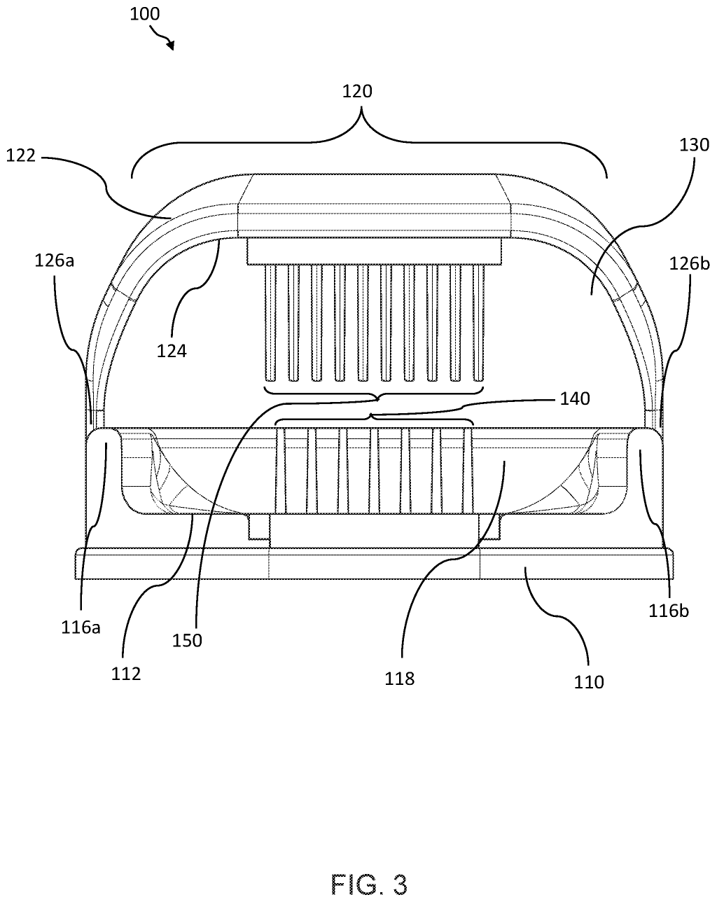

Referring now to FIG. 3, a back view is provided of the hands-free foot care and callous removal device 100 of FIG. 1. Base 110 comprises a top surface 112 and a bottom surface 110. A substantially horseshoe shaped portion 118 comprises two opposing side walls 116a and 116b. Bridge 120 comprises a top surface 122, a bottom surface 124, and two ends 126a and 126b, and the bottom surface of the bridge 124 and the top surface of the base 112 are separated by gap 130. Callous removal device 100 also comprises bottom brush plate 140 and top brush plate 150.

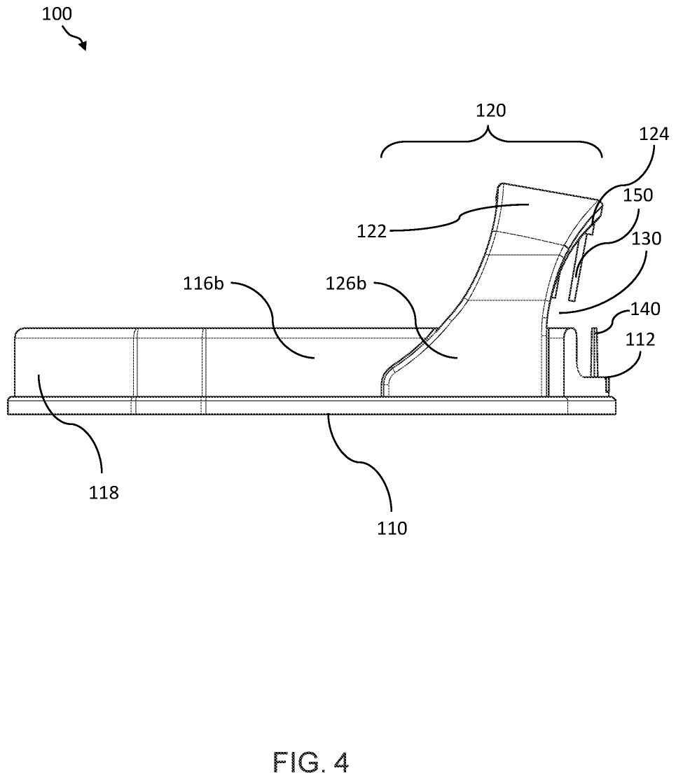

Referring now to FIG. 4, a side view is provided of the hands-free foot care and callous removal device 100 of FIG. 1. Bridge 120 comprises top surface 122 and end 126b attached to base 110. A substantially horseshoe shaped portion 118 comprises side wall 116b. Callous removal device 100 also comprises bottom brush plate 140 and top brush plate 150.

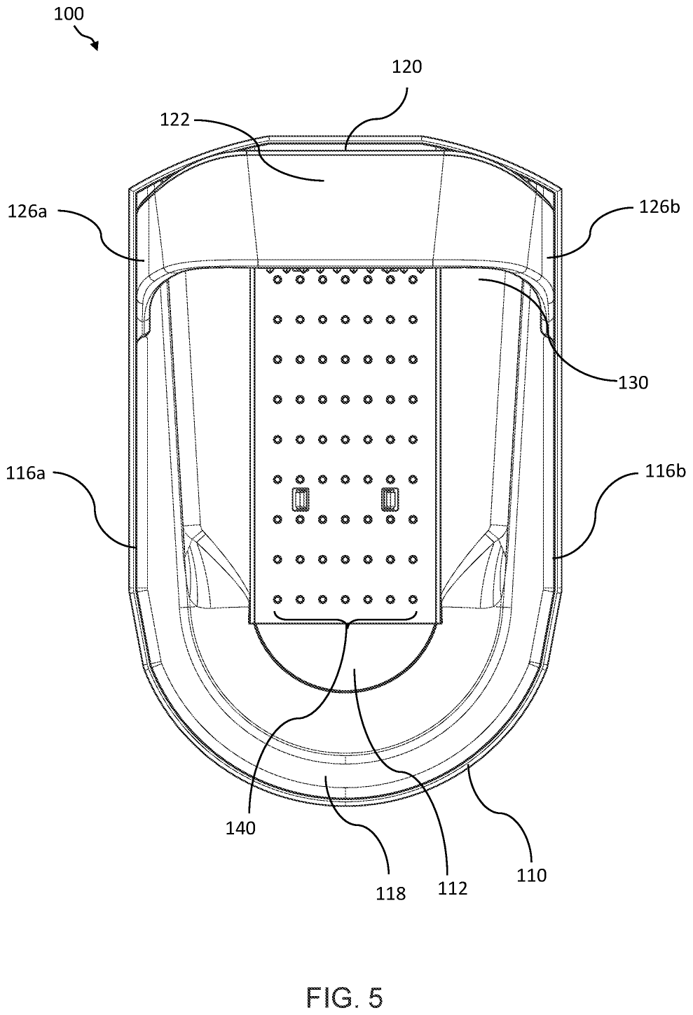

Referring now to FIG. 5, a top view is provided of the hands-free foot care and callous removal device 100 of FIG. 1. Base 110 comprises top surface 112. Bridge 120 comprises a top surface 122 and two ends 126a and 126b, wherein end 126a and end 126b are each attached to base 110. A substantially horseshoe shaped portion 118 comprises two opposing side walls 116a and 116b. Callous removal device 100 also comprises bottom brush plate 140 and top brush plate 150.

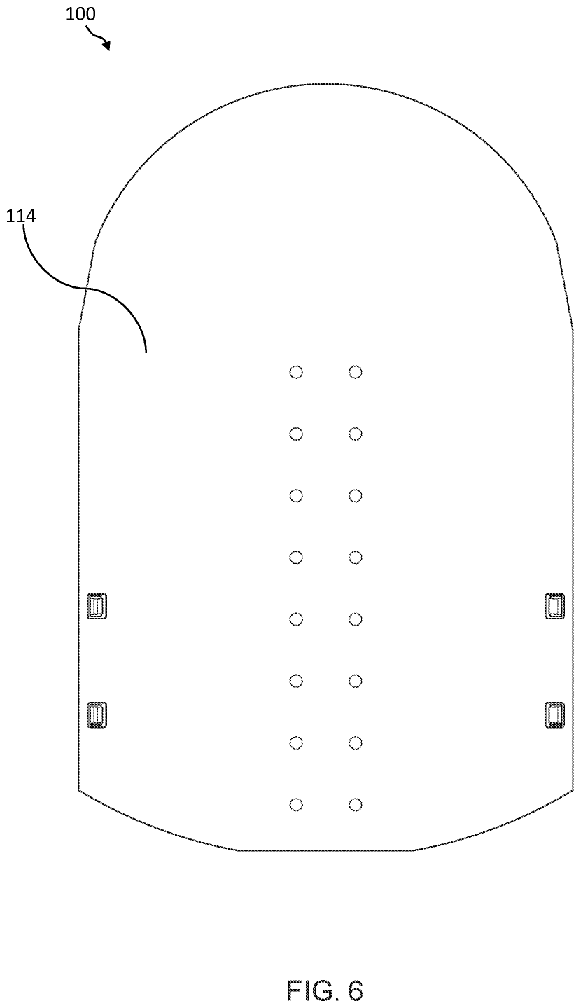

Referring now to FIG. 6, a bottom view is provided of the hands-free foot care and callous removal device 100 of FIG. 1. The base (not shown) comprises bottom surface 114.

In some embodiments, the top brush plate 150 and bottom brush plate 140 comprise bristles that are either non-movable (static, rigid, etc.) or movable (flexible, deflectable, bendable, etc.) relative to their points of attachment to the bottom surface of the bridge 124 or the top surface of the base 112.

In another embodiment, a method is provided for the hands-free use of foot care and callous removal device 100 to treat a user's foot, comprising: i) the user inserting their foot into the gap 130 to contact the bottom brush plate 140 and/or top plate 150 for hands-free cleaning and/or massaging of the foot; and/or ii) the user contacting the abrasive surfaces of the substantially horseshoe shaped portion 118 and opposing side walls 116a and 116b with the foot's top, sides, bottom, and/or heel for hands-free abrading of dead skin and/or callouses on the foot. In some aspects, the user has difficulty accessing surfaces of their feet for cleaning, massaging, or abrading callouses due to a chronic condition comprising arthritis, obesity, chronic back pain, or injury.

### Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A hands-free foot care and callous removal device, comprising:
a) a base comprising a top surface, a bottom surface, a substantially horseshoe-shaped portion comprising two opposing side walls 116a and 116b, wherein the substantially horseshoe shaped portion 118 and opposing side walls 116a and 116b comprise abrasive surfaces;
b) a bridge comprising a top surface, a bottom surface, and two ends, wherein each end is attached to the base, and wherein the bottom surface of the bridge and the top surface of the base are separated by a gap;
c) a bottom brush plate; and
d) a top brush plate;
wherein the gap, the bottom brush plate, and the top brush plate are configured to enable insertion of a user's foot into the gap to contact the bottom brush plate and/or the top brush plate for hands-free cleaning and/or massaging of the foot; and wherein the substantially horseshoe shaped portion and opposing side walls comprising abrasive surfaces are configured to enable the user to contact the abrasive surfaces with the foot's top, sides, bottom, and/or heel for hands-free abrading of dead skin and/or callouses on the foot.

2. The hands-free foot care and callous removal device of claim 1, wherein the top brush plate and the bottom brush plate each comprise bristles that are non-movable relative to their points of attachment to the bottom surface of the bridge or the top surface of the base.

3. The hands-free foot care and callous removal device of claim 1, wherein the top brush plate and the bottom brush plate each comprise bristles that are movable relative to their points of attachment to the bottom surface of the bridge or the top surface of the base.

4. A method for hands-free use of the hands-free foot care and callous removal device of claim 1 to treat a user's foot, comprising: i) a user inserting their foot into the gap to contact the bottom brush plate for hands-free cleaning and/or massaging of the foot; and/or ii) a user contacting the abrasive surfaces of the substantially horseshoe shaped portion and opposing side walls with the foot's top, sides, bottom, and/or heel for hands-free abrading of dead skin and/or callouses on the foot.

5. The method of claim 4, wherein the user has difficulty accessing surfaces of their feet for cleaning, massaging, or abrading callouses due to a chronic condition comprising arthritis, obesity, chronic back pain, or injury.
